(19) United States
(12) Patent Application Publication
(10) Pub. No.: US 2003/0220401 A1
(43) Pub. Date: Nov. 27, 2003

(54) Modulation of signal transduction

(76) Inventor: Gerhart Graupner, San Diego, CA (US)

Correspondence Address: FITCH EVEN TABIN AND FLANNERY, 120 SOUTH LA SALLE STREET, SUITE 1600, CHICAGO, IL, 60603-3406, US

(21) Appl. No.: 10/155,307

(22) Filed: May 22, 2002

(51) Int. Cl. A61K 31/203; C07K 14/47

(52) U.S. Cl. 514/559; 514/613; 530/350

(57) ABSTRACT

A polypeptide is identified as being functionally included in a signal transduction pathway having a biological effect. Contemplated polypeptides are different from a retinoic acid receptor, a retinoid X receptor, or a cellular retinoic acid binding protein; however, binding of the retinoid or retinoid metabolite leads to a modulation of the biological effect. In particularly contemplated methods, a retinoid or retinoid metabolite is administered to a cell or mammal in a concentration effective to modulate the biological effect.

This application claims the benefit of International Application PCT/US00/42233, filed Nov. 22, 2000, and the benefit of U.S. provisional application Serial No. 60/167,438, filed Nov. 23, 1999, the disclosures of both of which are expressly incorporated herein by reference.

### FIELD OF THE INVENTION

The field of the invention is modulation of signal transduction.

### BACKGROUND OF THE INVENTION

Many physiological and pathophysiological conditions can be influenced by presenting a cell, cell culture, or organism with a drug that directly or indirectly interferes with that condition. For example, drugs that directly interfere with a physiological and pathophysiological condition include enzyme inhibitors (e.g., penicillin inhibits bacterial transpeptidases, or Mevinolin™ inhibits 3-hydroxy-3-methylglutaryl coenzyme A reductase) or antisense nucleic acids (e.g., antisense DNA inhibit translation of viral genes). Although directly interfering drugs are often highly specific towards their target, their biological action is frequently limited to individual biochemical conversions or processes.

In order to modulate a plurality of biochemical reactions or physiological events, drugs that modulate signal transduction pathways may be employed, and numerous compositions and methods are known in the art to interfere with signal transduction pathways. For example, in one method of interfering with a signal transduction pathway, a signaling molecule (e.g., a cytokine or insulin) is added to a cell or organism. Adding signaling molecules is often advantageous, especially where exogenous addition to a system lacking the signaling molecule reconstitutes the physiologically normal level of the signaling molecule. However, addition of exogenous signaling molecules is frequently problematic, especially where the molecules are immunogenic peptides or peptide preparations with impurities and/or inhomogeneities.

Alternatively, receptors for the signaling molecules may be blocked or otherwise rendered functionally inactive. For example, a beta-blocker competitively inhibits binding of the natural signal adrenalin to beta-adrenergic receptors in the nervous system. Receptor blockers generally exhibit strong inhibition of their target receptors; however, they tend to inhibit non-target receptors, especially where the non-target receptors belong to the same family as the target receptor (e.g., various beta-blockers tend to block non-target beta-2 receptors).

In another example, elements within a signaling cascade may inhibit or prevent a signal from being transduced to the target compartment of a cell. A particularly promising element in a signaling cascade is the vascular endothelial growth factor (VEGF) receptor kinase, which specifically phopsphorylates its substrate in dependence of binding of VEGF to the VEGF receptor, and it has recently been shown, that VEGF kinase inhibitors effectively inhibit signaling in VEGF kinase associated pathways [Drevs J. et al. Effects of PTK787/ZK 222584, a specific inhibitor of vascular endothelial growth factor receptor tyrosine kinases, on primary tumor, metastasis, vessel density, and blood flow in a murine renal cell carcinoma model. Cancer Res 2000;60(17):4819-24]. However, even relatively low cross-reactivity with kinases other than VEGF kinases may potentially disrupt a plethora of non-targeted pathways due to the presence of various kinases in many other signaling pathways.

In a still further example, elements and processes at the end-point of a signaling cascade may inhibit or prevent the signal from being translated into a regulatory or other function in the cell. A typical example of “end-point inhibition” is the use of antisense nucleic acids that hybridize with a transcription product that is being formed in a response to the signal, or that form triple helices with a target sequence that is activated by the signal.

Although there are various methods of interfering with signal transduction pathways known in the art, all or almost all of them suffer from one or more disadvantages. Therefore, there is a need to provide novel methods for interfering with signal transduction pathways.

### SUMMARY OF THE INVENTION

The present invention is directed to compositions and methods of cell-specifically modulating a signal transduction pathway in a system. In one step, the signal transduction pathway is identified as functionally including a cellular polypeptide that binds a retinoid or retinoid metabolite, wherein the cellular polypeptide is not a retinoic acid receptor (RAR), retinoid X receptor (RXR), a vision pigment, nor a cellular retinoic acid binding protein (CRABP), and wherein binding of the retinoid or retinoid metabolite results in modulation of a biological effect modulated by the signal transduction pathway. In a further step, the retinoid or retinoid metabolite is administered to the system (e.g., a mammal, cell-, or tissue culture) in a concentration effective to modulate the biological effect.

In one aspect of the inventive subject matter, the retinoid has a cis-configuration and is preferably a 9-cis-retinoid, a 4-hydroxyphenyl-retinamide, or a 4-hydroxyphenyl-retinamide analog. Further contemplated retinoids and retinoid metabolites include various stereoisomers of retinylinositides, lipid-conjugated retinoids, sulfur-containing retinoids, and especially contemplated retinoid metabolites include a sulfated retinoid and S-adenosylretinoid.

In another aspect of the inventive subject matter, the cellular polypeptide comprises an ion channel, preferably with a specificity for K⁺, Ca⁺, Na⁺, or Cl−, and it is particularly preferred that the ion channel functionally cooperates with a sulfonylurea receptor (SUR). It is further especially contemplated that the ion channel comprises an adenosine triphosphate (ATP) gated potassium channel complex.

In a further aspect of the inventive subject matter, contemplated biological effects are amplified by binding of the retinoid or retinoid metabolite, and particularly contemplated biological effects include cell division, insulin secretion, cell growth, and arrythmia.

In a still further aspect of the inventive subject matter, contemplated retinoids have a bimodal effect, administration of the retinoid or retinoid metabolite at a first concentration modulates a first biological effect, and administration of the retinoid or retinoid metabolite at a second concentration modulates a second biological effect.

Various objects, features, aspects and advantages of the present invention will become more apparent from the following detailed description of preferred embodiments of the invention, along with the accompanying drawing.

### BRIEF DESCRIPTION OF THE DRAWING

FIGS. 1A-1D depict exemplary retinoids and retinoid metabolites.

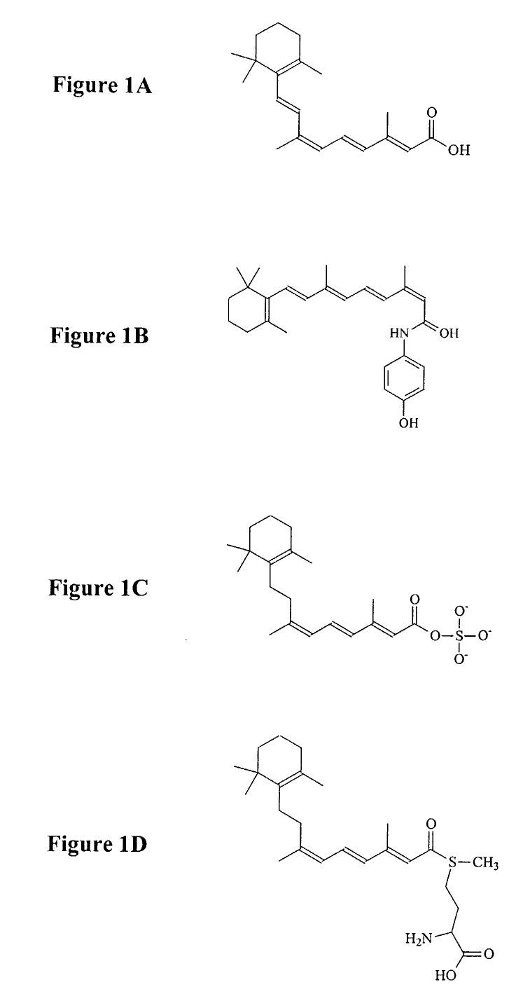

FIG. 2 depicts further exemplary structures of retinoids and retinoid metabolites.

FIGS. 3A-3D show confocal laser micrographs depicting co localization of apoptosis in cardiac conductance cells after administration of 9-cis-retinoic acid, and control.

FIGS. 4A-4D are graphs depicting expression levels of various enzymes and signal transduction markers after administration of 9-cis-retinoic acid compared to the corresponding control signal after administration of carrier.

### DETAILED DESCRIPTION

It is generally believed that retinoids interact in various signal transduction pathways via binding to the retinoid acid receptor (RAR) and/or the retinoid X receptor (RXR) [see e.g., Vitamins and Hormones, 49, 327-382 (1994)). For example, binding of retinoids to RAR and/or RXR has been demonstrated in signal transduction pathways involved in apoptosis [see e.g., Kastner, P. et al, The role of nuclear retinoid acid receptors in the regulation of gene expression; Vitamin A in health and disease, Marcel Decker, Inc. New York 189-238 (1994)]. It is further generally believed that the RAR and/or RXR act as nuclear transcription activators as dimers with a further RAR and/or RXR molecule or other nuclear transcription activator.

Surprisingly, the inventor discovered that retinoids and/or retinoid metabolites also bind to cellular polypeptides other than RAR and/or RXR, and that binding of the retinoids and/or retinoid metabolites to such cellular polypeptides results in a modulation of the biological effect in signal transduction pathways that functionally include such cellular polypeptides.

Consequently, it is contemplated that a method of cell-specifically interfering with a signal transduction pathway that controls a biological effect in a system may include one step in which the signal transduction pathway is identified as functionally including a cellular polypeptide that binds a retinoid or retinoid metabolite, wherein contemplated cellular polypeptides are polypeptides other than RAR, RXR a visual pigment, or CRABP, and binding of the retinoid or retinoid metabolite to such polypeptides results in a modulation of the biological effect. In a further step, the retinoid or retinoid metabolite is administered to the system in a concentration effective to modulate the biological effect. The term “binding to a cellular polypeptide” as used herein means that the cellular polypeptide retains a bound substance with a dissociation constant of less than 10⁻³/Mol, however, specifically excludes binding of a retinoid to a catalytically active site of an enzyme converting the retinoid to a retinoid metabolite.

In one particular aspect of the inventive subject matter, the inventor discovered that in vivo administration of 9-cis retinoic acid to mice resulted in reduced growth of cardiomyocytes, and specifically lead to apoptosis in cardiac neurons and cardiac conductive cells due to signal amplification in the corresponding mitochondrial signal transduction pathways in the cardiac neurons and cardiac conductive cells (see experimental data, infra).

It should be especially noted, that cardiac neurons and cardiac conductive cells, as well as cardiomyocytes contain significant amounts of RXR and RAR [see, e.g., Georgiades P, Brickell P M, Regulation of retinoid X receptor-gamma gene transcript levels in rat heart cells; Cell Biol Int 1998;22(6):457-63, and Kastner et al, Vitamin A deficiency and mutations of RXRalpha, RXRbeta and RARalpha lead to early differentiation of embryonic ventricular cardiornyocytes; Development 1997 December; 124(23):4749-58]. If in fact binding of the 9-cis retinoid or its metabolites to the RAR and/or RXR would lead to signal amplification in an apoptosis signal transduction pathway. A person of ordinary skill in the art would expect modulation of the signal transduction pathway in all of the cardiac neurons, cardiac conductive cells, and the cardiomyocytes. However, apoptosis was induced cell-specifically in the cardiac neurons and cardiac conductive cells, but not in cardiomyocytes. Therefore, it is contemplated that modulation of the apoptosis signal transduction pathway is not mediated by binding of the 9-cis retinoid to the RAR and/or RXR, and/or a heterodimeric complex including RXR, but by binding of the 9-cis retinoid to an alternative binding target.

With respect to the alternative (non-RAR/non-RXR) binding target of 9-cis retinoid or its metabolite, it should be appreciated that cardiac neurons, cardiac conductive cells, and cardiomyocytes are known to have mitochondrial ATP-gated K⁺ channels (see e.g., Light P E et al. Cardiovascular Res. 44, 356-369, 1999; Irisawa H et al., Cardiac pace making in the sino-atrial node, Physiol. Rev. 73, 197-227, 1993; Garlid K D, Cation transport in mitochondria-the potassium cycle, Biochirn. Biophys. Acta 1275, 123-126, 1996). It has further been proposed, that mitochondrial ATP-gated K⁺ channels are involved in apoptotic processes by increasing K⁺ influx and consequently increasing Ca²⁺ fluxes. Interestingly, it has been shown that the mitochondrial ATP-gated K⁺ channels are regulated in a protein complex by the sulfonylurea receptor (SUR), which has markedly distinct binding affinities towards its substrates when expressed as type 2 on cardiomyocytes and when expressed as type 1 on cardiac neurons cardiac/conductive cells (Aguilar-Bryan L and Bryan J, Molecular biology of adenosine triphosphate sensitive potassium channels, Endocr. Review 20, 101-135, 1999): Type 2 SUR on cardiomyocytes are known to have generally a lower affinity to various substrates (e.g., various sulfonylurea-based drugs) than type 1 SUR on cardiac neurons cardiac/conductive cells (Inagaki N et al., A family of sulfonylurea receptors determines the pharmacological properties of ATP-sensitive K⁺ channels, Neuron 16(5), 1011-1017, 1996). It has also been postulated, that cardiac conductive cells (i.e., cells that comprise the cardiac conductance system) contain type 1 SUR, and cardiomyocytes contain type 2 SUR. It is further known that binding of various substrates to SUR modulates the activity of ATP-gated K⁺ channels (Baukrowitz T et al. PIP2 and PIP as determinants for ATP inhibition of KATP channels, Science 282, 1141-1144, 1998; Fan Z and Makielski J C, Anionic phospholipids activate ATP-sensitive potassium channels, J. Diol. Chem 272, 5388-5395, 1997; Heron L et al., Human alpha-endosulfine, a possible regulator of sulfonylurea-sensitive KATP channel: molecular cloning, expression and biological properties, Proc. natl. Acad. Sci. U.S.A. 95, 8387-8391, 1998; Holemans et al., Interaction of fluorescein derivatives with glibenclamide binding sites in rat brain, Neuroscience Lett. 183, 183-186, 1995). Based on observations of the inventor (unpublished data), but not wishing to be bound to a particular hypothesis or theory, it is contemplated that retinoids and their metabolites specifically bind to SUR and/or the SUR-K⁺ channel complex, and it is further contemplated that such binding modulates (e.g., up-regulates) the activity of the ATP-gated K⁺ channel.

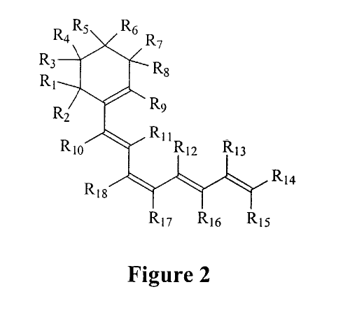

In further aspects of the inventive subject matter, it is contemplated that in alternative methods of cell-specifically interfering with a signal transduction pathway the system need not be limited to a mouse, and appropriate systems include in vivo and in vitro systems. For example, suitable in vitro systems include cell and tissue cultures, wherein the cells may be derived from a live specimen (e.g., biopsy), a secondary cell culture, or a thawed cell or tissue sample. Particularly contemplated cells are mammalian cells, however, non-mammalian vertebrate and invertebrate cells are also contemplated. Especially contemplated in vivo systems, include mammals (and particularly human), non-mammal vertebrates, and invertebrates (e.g., yeasts). With respect to the retinoid, it is contemplated that all known retinoids are suitable for use in conjunction with the teachings presented herein, and exemplary retinoids are described in Chemistry and Biology of Synthetic Retinoids (Marcia Dawson, William H. Okamura (Editor), CRC Pr; ISBN: 0849347971), Retinoids: The Biochemical and Molecular Basis of Vitamin A and Retinoid Action (Heinz Nau (Editor), William S. Blaner (Editor); Springer Verlag; ISBN: 3540658920), or Retinoids (Maria A. Livrea, Lester Packer (Editor); Marcel Dekker; ISBN: 0824787587), all of which are incorporated by reference herein. However, it is preferred that the retinoid comprises at least one cis-configuration, and it is particularly contemplated that the cis-configuration is a 9-cis configuration (e.g., 9-cis retinoic acid). Exemplary retinoids are depicted in FIG. 2, in which R₁-R₁₈ are independently selected from H, Halogen, alkyl, alkenyl, alkynyl, aryl, alkraryl, all of which may independently 10 further comprise a halogen, a functional group (e.g., a CHO, COOH, NO₂, NO, NH₂, NH, SO₂, SO, PO⁴), a polar and/or hydrophilic group, including mono-, di-, and polysaccharides, a non-polar and/or hydrophobic group, including fatty acids, lipids, steroids, etc, and an amino acids and/or an amino acid derivatives. Contemplated R₁-R₁₈ may further comprise heteroatoms, which may be in various positions of R₁-R₁₈, including pending or in the backbone. Where stereoisomeric or chiral variations of contemplated retinoids exist, all chemically reasonable configurations (of R, S, cis, and trans) are contemplated. For example, suitable retinoids need not be limited to the depicted 9-cis configuration, but may also include all-trans, 9-cis-11-cis-13-transconfiguration, etc. However, the term “retinoid” as used herein particularly excludes retro-retinoids as described in Retro-Retinoids in Regulated cell growth and death, O'Connel et al., J. Exp. Med 1996, 184: 549-555.

In further contemplated aspects, retinoid analogs are contemplated as suitable alternative retinoids. The term “retinoid analog” as used herein refers to any molecule that displays an activity conventionally ascribed to retinoic acid derivatives as summarized in U.S. Pat. No. 6,034,242 to Vuligonda et al. (Mar. 7, 2000), which is incorporated by reference herein. An especially contemplated retinoid analog is 4-hydroxyphenyl-retinamide or a 4-hydroxyphenyl-retinamide analog, as described in U.S. Pat. No. 6,117,845, which is also incorporated by reference herein. Further contemplated retinoid analogs especially include known bicyclical spaced conformationally constrained ligands of RXR, and particularly include RXR agonists such as LG1069, and compounds described by Benoit et al. (RAR-independent RXR signaling induces t(15; 17)leukemia cell maturation EMBOJ(1999)18(24), 7011-7018).

It should be particularly appreciated that the retinoid or retinoid analog may also be metabolized in one or more biochemical (e.g., enzymatic) or thermo dynamical (e.g., thermal isomerization) conversion to form a retinoid metabolite or retinoid analog metabolite. There are numerous metabolic conversions in which the retinoid and/or retinoid analog may be metabolized, and all of the known metabolic conversions are contemplated. For example, a metabolic conversion may include addition of a chemical function or group, including polar, charged, lipophilic or hydrophilic groups, sulfur, nitrogen, or oxygen containing groups, etc. Further contemplated groups particularly include sulfates, thiols, and adenosyl methionine, but also saccharides (e.g., hexoses and/or pentoses) and heterocyclic compounds (e.g., bases), which may further be linked to sugars and/or alcohols. Consequently, it is contemplated that retinoid metabolites include a sulfated retinoid and S-adenosylretinoid, and various stereoisorneric forms of retinolinositides.

It should still further be appreciated, that the cellular polypeptide to which the retinoid or retinoid metabolite binds need not be restricted to a SUR-ATP gated K⁺ channel complex, and alternative polypeptides include ion channels with a selectivity for calcium ions, sodium ions, or chloride ions. It is especially contemplated that the ion channel functionally cooperates with an SUR. Alternative further contemplated polypeptides include a complex comprising an inward rectifier potassium channel HERG with a member of the MinK family of regulatory transmembrane peptides. The term “ion channel functionally cooperates with an “SUR” as used herein means that the activity of the ion channel is directly or indirectly influenced by the SUR. The term “directly influenced” as used herein means that the SUR is physically coupled to the ion channel, and that binding of the retinoid to the SUR and/or SUR-ion channel complex influences the activity of the ion channel. The term “indirectly influenced” as used herein means that the SUR is not physically coupled to the ion channel, and that the activity of the ion channel may be modulated by at least one intermediary molecule between the SUR and the ion channel complex when the retinoid or retinoid metabolite binds to the SUR. Still further contemplated alternative cellular polypeptides include membrane transport proteins for cellular detoxification, and particularly include members of the family of mgm and PgP.

Consequently, it is contemplated that alternative biological effects (i.e., biological effects other than apoptosis) include all biological effects that are mediated by a signal transduction pathway, which includes an ion channel and/or an SUR. For example, it is known that insulin secretion of neuroendocrine cells is at least partly regulated by the activity of a K⁺ ion channel and/or SUR (see e.g., Gribble F et al. Tissue specificity of sulfonylureas, Diabetes 47, 1412-1418, 1998). Similarly, various neuroendocrine disorders that are at least partly linked to activity of a K⁺ ion channel and/or SUR are contemplated (see e.g. Shyng S L et al. Functional analyses of novel mutations in the sulfonylurea receptor 1 associated with persistent hyperinsulinemic hypoglycemia of infancy, Diabetes 47, 1145-1150, 1998). Furthermore, contemplated diseases include cystic fibrosis, which is known to involve malfunction of a chloride ion channel in the pathogenesis. In another example, contemplated biological effects may also include cell growth, cell division, and arrythmia, all of which are contemplated to be modulated by a signal transduction pathway that includes an ion channel and/or SUR (Malhi H et al, KATP channels regulate mitogenically induced proliferation in primary rat hepatocytes and human liver cell lines, J. Biol. Chem. 275, 26050-7, 2000; Noble D. The ionic basis of the heartbeat and of cardiac arrythmias, in: B N Singh, H J J Wellens, M Hiraoka (eds): Electropharmacological Control of Cardiac Arrhythmias, Mount Kisco, N.Y., Futura Publ. 1994, p. 3-20, and following chapters). In a still further example, where the retinoid binding motif comprises an ATP binding cassette (ABC), it is contemplated that administration of the retinoid may include modulation of a drug resistant phenotype in cancer cells (e.g., via interaction with the ABC in PgP or mdm). It should further be appreciated that the modulation of the biological effect may comprise amplification or reduction of the biological effect.

It is still further contemplated that the retinoid may also bind to a cellular polypeptide other than an ion channel, and suitable alternative polypeptides include transmembrane proteins, membrane associated proteins, cytosolic proteins, proteins associated with or located within cellular compartments, including mitochondria, endoplasmatic reticulum, endosomes, and the nucleus. For example, particularly contemplated alternative cellular polypeptides include inositide binding polypeptides such as PI₃-kinase, protein kinase B, PI₄-kinase, PI_{4,5}- kinase, and inositol phosphate receptor isoforms. Also contemplated are P2X ion channels, which are regulated by extracellular ATP, bcl-x and bcl-2. ITP/IDP and GTP/GDP binding proteins are particularly preferred, and binding of cis-retinoids is especially preferred over binding of all-trans retinoids. However, particularly excluded from appropriate alternative cellular proteins are RAR, RXR, and CRABP-1, CRABP-11, and retinoid isomerases in the vision pathway (Wald, C., Science 162, 230-239 (1968)). It should be recognized that alternative cellular polypeptides are not nuclear polypeptides that act alone or in combination with another proteins as a transcription factor.

Consequently, a ligand-polypeptide complex is contemplated, that has a ligand comprising at least one of a retinoid and a retinoid metabolite, and that further has polypeptide other than a retinoic acid receptor, a retinoid X receptor, and a cellular retinoic acid binding protein, wherein the polypeptide binds the ligand with a dissociation constant of less than 10⁻³/Mol, and wherein binding of the ligand to the polypeptide results in a modulation of a biological effect in a signal transduction pathway.

With respect to the administration of the retinoid or retinoid metabolite, it is contemplated that a particular system will typically determine the particular administration. For example, where the system is a cell or tissue culture, the administration will typically comprise admixing a retinoid-containing solution to the cell or tissue culture. On the other hand, where the system is a human, mammal, or other animal, suitable administrations include oral or parenteral administration, injection, infusion, topical or transdermal application, etc. Likewise, it should be appreciated that the schedule of administration will predominantly depend on the particular system and biological effect, and it is therefore contemplated that the schedule may vary considerably. For example, while in some cell cultures a single administration will produce the desired modulation of the biological effect, administrations to a human may require multiple administrations to obtain the desired modulation of the biological effect.

Similarly, the dose of the retinoid or retinoid metabolite may vary substantially, and it is contemplated that the dose is generally within the range of several micrograms and several grains. However, it is preferred that the concentration of the retinoid in the system is between about 1 micromolar and about 10 millimolar. In further contemplated aspects of the inventive subject matter, it should be recognized that administration of contemplated retinoids or retinoid metabolites has a bimodal effect with respect to the amount of administered retinoids and retinoid metabolites. Among other effects, the inventor has observed that while administration of moderate amounts (i.e., 3-10 mg/kg) of 9-cis retinoic acid to mice leads to reduced growth of cardiomyocytes, cardiac neurons, and cardiac conductive cells, administration of higher amounts (i.e., 20 mg/kg and higher) of 9-cis retinoic acid leads to reduced growth of cardiomyocytes, and apoptosis of cardiac neurons and cardiac conductive cells. Thus, it is contemplated that retinoids and/or retinoid metabolites exhibit a bimodal activity. Consequently, it is contemplated that administration of a retinoid and/or retinoid metabolite at a first concentration has a modulation of a first biological effect, and administration of the retinoid and/or retinoid metabolite at a second concentration has a modulation of a second biological effect. Particularly contemplated first biological effects include reduction of growth in cardiomyocytes, and particularly contemplated second biological effect include induction of apoptosis in a cardiac neurons and cardiac conductive cells.

Experimental Data

Animal Experiments

1-week-old litters of CL57/b1 mice (around 3 g body weight) were divided into groups of five to 10 animals, to be treated with all-trans-RA in carrier, 9-cis-retinoic acid (RA) in carrier, or carrier (canola oil, Sigma). Every treatment group was assigned to one nursing mother. Groups larger than IO pups were avoided, to exclude limited feeding frequency as experimental variable. A retinoid preparation for gavage was formulated such that every animal received a maximum of 150 μl volume containing 40 mg/kg all-trans RA in carrier, 12 mg/kg or 20 mg/kg 9-cis-RA in carrier, or carrier alone. DMSO or ethanol content was limited to 0.1%. Pups were gavage-fed from a tuberculin syringe connected with a bent vein catheter. The strong suckling reflex facilitated delivery of the viscous and odorous formulation. Depending on the territorial protection behavior of the mother, pups were briefly rolled in cage stray after gavage to prevent triggering maternal infanticide behavior through unusual smell of the pups. For two hours after gavage, pups were carefully monitored to prevent maternal infanticide. Every 24 h, the weight was recorded.

For uptake kinetics, pairs of treated pups were sacrificed by decapitation after 1 h, 2 h, 4 h, 8 h, 16 h, 24 h, 36 h and 72 h. Organs were removed under the dissection microscope and frozen in liquid nitrogen. Hearts were dissected under the surgical dissection microscope; tissues were divided into atria, ventricles, and enriched in conductance system (a tissue preparation containing the ventricular septa with the Purkinje fibers and the atrial region between the aorta and the pulmonal artery (outflow tract) with the sinus node). For immunohistology, pups were sacrificed after 72 h, hearts were rinsed in PBS, weighed and fixed in 4% para-formaldehyde in PBS before embedding in OCT for standard cryostat procedures. If pups were on the brink of death (typically between 36 and 72 h after gavage), they were sacrificed, and hearts were collected for confocal microscopy or HPLC.

Immunohistology

For confocal microscopy, cryostat sections were first shined for apoptotic DNA strand breaks according to the TUNEL protocol (Apoptag kit, Oncor. Gaithersburg) and counterstained with rabbit anti-connexin37 antibody or rabbit anti-connexin40 antibody for conductance cells, or with rabbit anti-myosin antibody for cardiac myocytes. Immunodetection of connexin isoforms 40 and 43 has been established as a useful method to detect cells of the cardiac conductance system in rodents (Gourdie et al., The spatial distribution and relative abundance of gap-junctional connexin40 and connexin43 correlate to functional properties of components of the cardiac atrioventricular conducting system, J. Cell Science 105 (Pt 4) 985-991, 1993). Because of the relatively weak signal of connexin40, the connexin isoform 37 can be used as an equivalent marker for the conductance system in the mouse model (Willecke K et al, 1991; Willecke K, personal communication). The combination of rabbit anti-Neuron-specific enolase with propidium iodide followed published procedures (Current Protocols in Molecular Biology) and was used to identify cardiac neurons with advanced apoptotic condensation of nuclei. Procedures of immunohistology and image analysis are further described in detail in Graupner, WO 00/53236.

The mechanism of apoptosis was investigated using commercially available primary antibodies of highest purity, at dilutions recommended by the manufacturer, against IGF-I, IGF-II, the entire set of IGFbinding proteins (IGFBPI-5), IGF-R, mitochondrial Mn-dependent superoxide dismutase Mn-SOD, and cytoplasmic Cu/Zn-dependent superoxide dismutase Cu/Zn-SOD. Cellular phenotypes were identified with counterstain using cardiac myosin, actin, connexin37, connexin40, and connexin43 antisera. Confocal images were captured and processed for quantitative analysis by Zeiss LSM-300 software. Briefly, the pixel intensities per unit area amounting to channel background were determined for each antibody/secondary antibody combination and subtracted from the experimental pixel intensities. Pixel intensities per fluorescent channel in regions of high signal were normalized to pixel intensity per unit area, as were pixel intensities in control regions. At least five independent regions of similar size were analyzed per tissue type and retinoid treatment condition. Averaged pixel intensities per unit area upon 9-cis retinoid induction were expressed as multiples of averaged pixel intensities per unit area in the absence of 9-cis retinoid induction (fold induction+/−s.e.m.).

The mechanism of apoptosis was investigated using commercially available primary antibodies of highest purity, at dilutions recommended by the manufacturer, against IGF-I, IGF-II, the entire set of IGFbinding proteins (IGFBP 1-5), IGF-R, mitochondrial Mn-dependent superoxide dismutase Mn-SOD, and cytoplasmic Cu/Zn-dependent superoxide dismutase Cu/Zn-SOD. Cellular phenotypes were identified with counterstain using cardiac myosin, actin, connexin37, connexin40, and connexin43 antisera. Confocal images were captured and processed for quantitative analysis by Zeiss LSM-300 software.

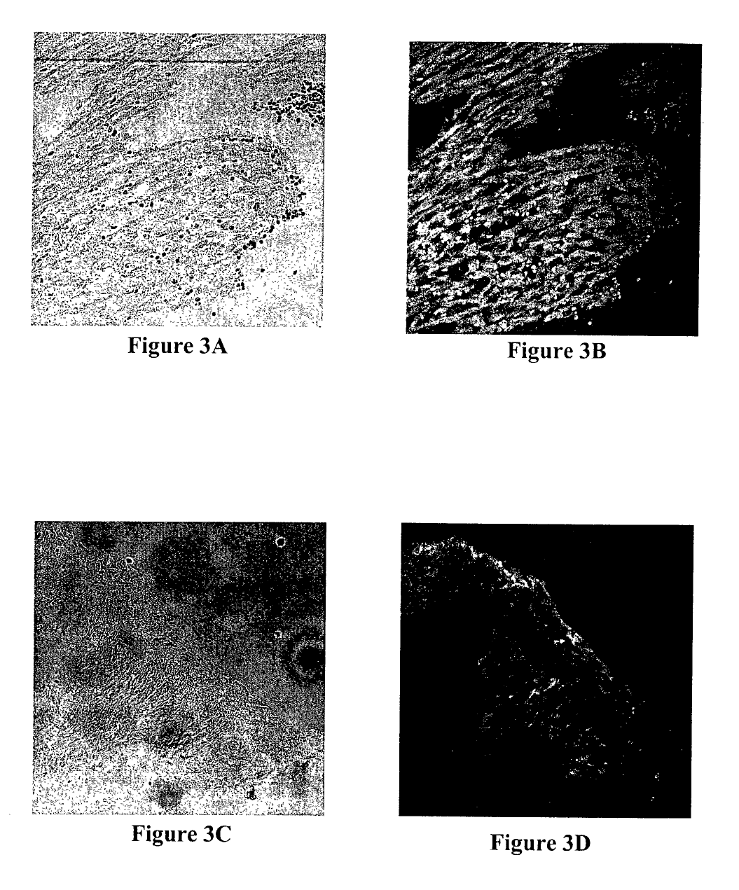

As an example, paired confocal laser micrographs from perinatal mouse heart sections after administration of 20 mg/kg 9cis-RA, or after administration of carrier, are presented in FIGS. 3A-3D. To correlate apoptotic events with a cellular phenotype, apoptotic cells were identified in the transmitted light mode of the confocal microscopy, DIC images were recorded, and matching images were scanned in the fluorescent mode, permitting accurate overlay images.

FIG. 3A illustrates the occurrence and distribution of apoptotic cells in the outflow tract region of a mouse heart after treatment with 20 mg/kg 9cis-RA. FIG. 3B is the corresponding fluorescence image that shows the staining of apoptotic cells with the mouse conductance cell marker connexin37. FIG. 3C illustrates the absence of apoptotic cells in the outflow tract region of a mouse heart after treatment with carrier. FIG. 3D is the corresponding fluorescence image that shows the presence of cells stained with the mouse conductance cell marker connexin37 in the same section.

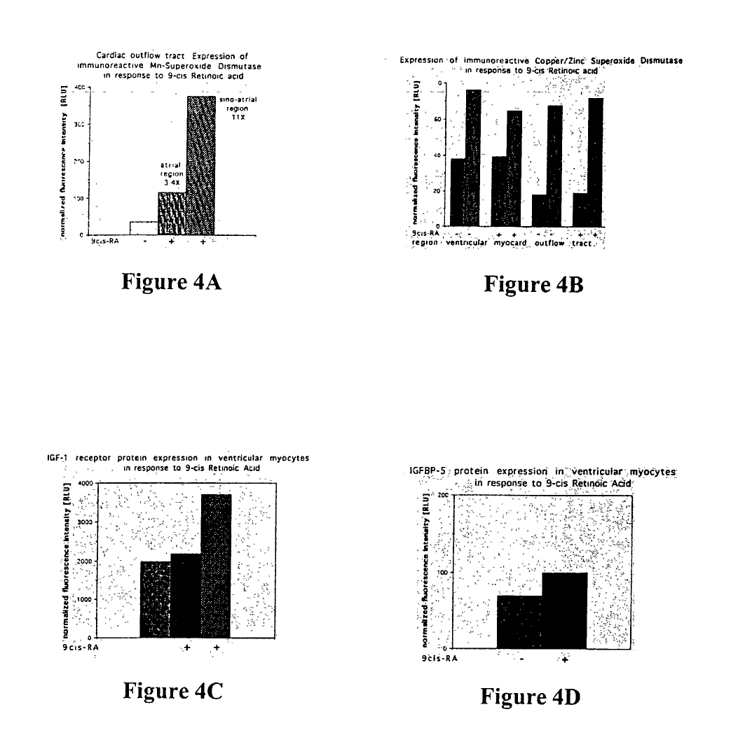

Further examples of quantitative confocal analysis of perinatal mouse heart sections are presented in graph form in FIGS. 4A-4D. Pixel intensities (per unit area) amounting to channel background were determined for each antibody/secondary antibody combination. This background signal was subtracted from pixel intensities per unit area observed under different experimental conditions, or under control conditions. If substantially different levels of marker expression were observed in distinct areas of a supposedly identical cellular environment under the same experimental condition, such signals were grouped and presented in a separate bar (e.g. in FIG. 4B for signals from Cu/Zn-SOD levels, and in FIG. 4C for IGF-Receptor levels). At least five independent regions of similar size were analyzed per tissue type and retinoid treatment condition; background-corrected, area-normalized pixel intensity averages were determined and are presented as relative pixel intensities. In FIG. 4A, averaged pixel intensities per unit area upon 9cis retinoid induction were expressed as multiples of averaged pixel intensities per unit area in the absence of 9cis retinoid induction (fold induction).

FIG. 4A quantifies the up regulation of immunoreactive Mn-dependent Superoxide Dismutase (Mn-SOD) in mitochondria of cells in the cardiac outflow tract after administration of 20 mg/kg 9cis-RA as fold induction of the corresponding control signal after administration of a carrier. FIG. 4B shows no significant change in expression of Cu/Zn-dependent Superoxide Dismutase in perinatal murine cardiac myocytes and in cells of the perinatal murine cardiac outflow tract after administration of 20 mg/kg 9cis-RA in comparison to the corresponding control signal after administration of the carrier; two markedly different levels of Cu/Zn SOD expression can be observed in either retinoid-treated or I control hearts. FIG. 4C shows that two different levels of IGF-I receptor expression can be observed in perinatal murine ventricular myocytes after administration of 20 mg/kg 9cis-RA in comparison to the corresponding control signal after administration of the carrier; one compartment of cardiomyocytes contains IGFR levels that are the same as in control animals, another compartment of cardiomyocytes contains significantly elevated levels of IGFR. FIG. 4D describes the marginal increase in IGF-binding protein 5 expression in perinatal murine cardiomyocytes after administration of 20 mg/kg 9cis-RA in comparison to the corresponding control signal after administration of the carrier.

HPLC Analysis

Tissues collected for retinoid analysis were dispersed by two rounds of sonication. The slurry was doted with a known amount of synthetic retinoid to correct for variable extraction efficiencies between different samples. Extraction steps followed published procedures (see e.g. Biesalski H K, Comparative assessment of the toxicology of vitamin A and retinoids in man, Toxicology 57, 117-161, 1989). After evaporation of the extraction solvents, the crude retinoid fraction was resuspended in HPLC buffer, injected on a RPC column and eluted with decreasing polarity in an isocratic gradient. The elution profiles were recorded and analyzed with system software. The positions of retinoic acid isoforms were identified, and absolute amounts of individual retinoids per mg tissue could be calculated.

Results

The surprising bimodal effect, namely sudden death of mouse pups after 9cis-RA gavage of nonmutagenic amounts and the cardiac growth arrest, bears resemblance to growth factor withdrawal.

After application of high concentrations of 9cis-RA (20 mg/kg), a strong correlation was found between apoptosis and cells immunoreactive with conductance cell markers. The argument of immunoreactivity is supported by the topography of the cells within the heart, consistent with the phenotype of conductance cells.

The results of connexin37 staining (FIGS. 3A-3D) and connexin40 staining (not shown) were consistent. There was no correlation between apoptosis and conductance cell type after either high all-trans-RA application (40 mg/kg), lower concentrations of 9cis-RA (less than 10 mg/kg), or carrier application. No colocalization of TUNEL signal was found in cardiac myocytes after 9cis-RA treatment at either 20 mg/kg or less than 10 mg/kg, although the size of the myocardium was reduced. The myocardial growth stunting seen at lower concentrations of 9cis-RA (less than 10 mg/kg) approached complete growth arrest around 10 mg/kg 9cis-RA, in contrast to the limited effect (less than 30% growth reduction) caused by substantially higher doses (40 mg/kg) of at RA.

Detailed confocal analysis of IGF pathways, known to be of primary significance in muscle cell growth regulation, in cardiac cell types reveals no indication for a connection between interruption of upstream IGF signaling and either myocardial growth stunting or tissue-specific apoptosis of conductance cells. IGF ligand levels, IGF receptor levels, and modulatory binding protein levels (IGFBP 1-5) were examined. While IGFBP5, a known negative regulator of IGF-I function, is marginally up-regulated in cardiac myocytes after 9cis-RA treatment (FIG. 4C), the scope of the response is unlikely to account for the full picture of the growth arrest. Surprisingly, the level of IGF-Receptor was found to be unchanged in one population of the myocardial myocytes, and even significantly up-regulated in another population of cardiomyocytes (FIG. 4D).

No evidence was found for involvement of the fas/TNF-Receptor pathway in conductance cell apoptosis.

Another observation with significant ramifications is the substantial activation of pathways metabolizing reactive oxygen species in cardiac tissue after administration of 20 mg/kg of 9cis-RA. First, upon addition of H₂O₂ in concentrations typically used to quench endogenous peroxidase activity, remarkable amounts of gas bubbles were released from large areas of myocardial cells in 9cisRA-treated tissue sections, but not in sections from animals treated with either all-trans-RA or carrier. This result is indicative for vastly increased amounts of endogenous peroxidase activity upon administration of high levels of 9cis-RA, and would be consistent with increased amounts of endogenous reactive oxygen species, which are not detectable by the methodologies applied. The pressure of the gas released led to detachment of several tissue sections. By qualitative judgment, the amount of gas released was too high to be stored in, or produced by, conductance cells which are of very low abundance compared to myocytes. Second, very high up-regulation of MnSOD protein signal was detected in connexin-positive cells of the cardiac outflow tract; the signal of 3.4 fold up-regulation localizes to the region of the atrium surrounding the sino-atrial node; the signal of 11 fold up-regulation localizes most likely to the area of the sino-atrial node proper. Thus, apoptosis occurs in cells that have initiated a program to counteract the damaging effects of reactive oxygen species through up-regulation of MnSOD at the protein level. Conversely, cardiac myocytes survive well with high levels of endogenous peroxidase activity and without elevated levels of enzymes protecting against reactive oxygen species. Thus, retinoid-induced apoptosis and enzymatic pathways producing and eliminating reactive oxygen are separate phenomena not causatively linked in the execution of programmed cell death in perinatal cardiac tissue.

Blood/Plasma, ventricles, atria and conductance cell region all contain the same types of retinoic acid isoforms in similar concentrations. The pharmacokinetic behavior of retinoic acid isoforms upon administration by gavage is substantially the same across all the cardiac tissues investigated. Thus, the HPLC data clearly refute the hypothesis that highly preferential uptake of retinoid into conductance cells, but not into cardiomyocytes, could account for the induction of tissue-specific apoptosis.

It should be especially appreciated that none of the well-known mechanisms and pathways, such as IGF signaling for cardiac growth, or generation of reactive oxygen species by retinoids, gives a satisfactory explanation for the observed bimodal action of 9cis-RA.

Thus, it should be recognized that contemplated methods particularly lend themselves to development and improvement of anti-diabetic drugs with reduced cardiac side-effects by selecting retinoid and retinoid analogs/derived compositions (which are preferably devoid of RXR agonist activity) that bind to SUR1, but not to SUR2. Likewise, it is contemplated to utilize retinoids and retinoid analogs/derived compositions to selectively modulate the function of SUR2 and thus generate novel compositions of channel-selective anti-arrythmic compounds. Another contemplated use of retinoids and retinoid analogs/derived compositions is the selective modulation of transport proteins for cellular detoxification, with the purpose to increase the susceptibility of highly drug-resistant tumors to antiproliferative therapy.

Thus, specific embodiments and applications of cell specific modulations of signal transduction pathways have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A method of cell-specifically interfering with a signal transduction pathway that controls a biological effect in a system, which method comprises:
identifying the signal transduction pathway as functionally including a cellular polypeptide that binds at least one of a retinoid, a retinoid analog and a retinoid metabolite;
wherein the cellular polypeptide is a polypeptide other than a retinoic acid receptor, a retinoid X receptor, and a cellular retinoic acid binding protein, and wherein binding of the at least one of a retinoid, a retinoid analog and a retinoid metabolite results in a modulation of the biological effect; and
administering the at least one of a retinoid, a retinoid analog and a retinoid metabolite to the system in a concentration effective to modulate the biological effect.

2. The method of claim 1 wherein the system comprises at least one of a cell culture and a tissue culture.

3. The method of claim 1 wherein the system is a mammal.

4. The method of claim 1 wherein the at least one of a retinoid, a retinoid analog and a retinoid metabolite has a cis-configuration.

5. The method of claim 4 wherein 9-cis-retinoic acid is administered.

6. The method of claim 1 wherein a 4-hydroxyphenyl-retinamide or a 4-hydroxyphenyl-retinamide analog is administered.

7. The method of claim 1 wherein a retinoid metabolite comprising a sulfur atom is administered.

8. The method of claim 1 wherein the cellular polypeptide comprises an ion channel.

9. The method of claim 8 wherein the ion channel is specific for an ion selected from the group consisting of a potassium ion, a calcium ion, a chloride ion, and a sodium ion.

10. The method of claim 8 wherein the ion channel functionally cooperates with at least one of a sulfonylurea receptor and a member of a Min-K channel family.

11. The method of claim 10 wherein the ion channel comprises an adenosine triphosphate gated potassium channel complex.

12. The method of claim 1 wherein the cellular polypeptide comprises a membrane transport protein for cellular detoxification.

13. The method of claim 1 wherein the biological effect being modulated comprises cell division, insulin secretion, cell growth, arrhythmia or drug resistance of a tumor cell.

14. The method of claim 1 wherein the step of administering comprises at least one of an oral administration and a parenteral administration.

15. The method of claim 1 wherein the modulation of the biological effect comprises amplification of the biological effect.

16. The method of claim 1 wherein administration of the at least one of a retinoid, a retinoid analog and a retinoid metabolite at a one concentration effects modulation of a first biological effect, and wherein administration of the at least one of a retinoid, a retinoid analog and a retinoid metabolite at a different concentration effects modulation of a second biological effect.

17. The method of claim 16 wherein the first biological effect comprises reduction of growth in a first cell, and wherein the second biological effect comprises induction of apoptosis in a second cell.

18. An isolated ligand comprising at least one of a retinoid, a retinoid analog and a retinoid metabolite, which will complex with a particular polypeptide, said particular polypeptide being other than a retinoid-metabolizing enzyme, a retinoic acid receptor, a retinoid X receptor, and a cellular retinoic acid binding protein, wherein the polypeptide binds the ligand with a dissociation constant of less than 10⁻³ Mol, and wherein binding of the ligand to the polypeptide results in a modulation of a biological effect in a signal transduction pathway in an animal or in an animal cell culture or tissue culture.

19. The isolated ligand of claim 18 wherein the ligand comprises 9-cis retinoic acid, and the particular polypeptide comprises an ion channel.

20. The isolated ligand of claim 18 wherein the ligand is a synthetic retinoid or retinoid analog.

21. The isolated ligand of claim 20 which complexes with a particular polypeptide comprising an ion channel or an ion channel plus a regulatory peptide.
